(19) United States
(12) Patent Application Publication
(10) Pub. No.: US 2020/0215158 A1
(43) Pub. Date: Jul. 9, 2020

(54) Multifunctional, Self-Assembled Aptamer-Nanomedicine Compositions and Methods for treating Cancer

(71) Applicant: The Methodist Hospital, Houston, TX (US)

(72) Inventor: Youli ZU, Bellaire, TX (US)

(21) Appl. No.: 16/703,844

(22) Filed: Dec. 4, 2019

Related U.S. Application Data
(60) Provisional application No. 62/775,386, filed on Dec. 4, 2018.

(51) Int. Cl. A61K 38/17; A61K 31/704; A61K 31/7105; A61P 31/00; A61P 35/00

(52) U.S. Cl. CPC A61K 38/1774; A61K 31/704; A61P 35/00; A61P 31/00; A61K 31/7105

(57) ABSTRACT

Disclosed are methods and compositions for diagnosis, imaging, and treating one or more mammalian diseases, including, for example, treatment, prophylaxis, and/or amelioration of one or more symptoms of a human cancer. In particular, compositions and methods are provided for the treatment and amelioration of one or more symptoms of lymphoma, and in exemplary embodiments, for treatment of anaplastic large cell lymphoma in humans.

### CROSS-REFERENCE TO RELATED APPLICATIONS

The present application claims priority to U.S. Provisional Patent Appl. No. 62/775,386, filed Dec. 4, 2018 (pending; Atty. Dkt. No. 37182.238PV01); the contents of which is specifically incorporated herein in its entirety by express reference thereto.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present disclosure relates to the fields of medicine, molecular biology, and specifically to the treatment of cancer. The disclosure provides compositions and methods for treatment and amelioration of one or more symptoms of lymphoma, and particularly for treatment of anaplastic large cell lymphoma (ALCL).

### Deficiencies in the Prior Art

Chemotherapy is the mainstream treatment of anaplastic large cell lymphoma (ALCL). However, chemotherapy can cause severe adverse effects in patients because it is not ALCL-specific. What is lacking is an improved method for treating ALCL that improves patient outcomes and produces less side effects than current standards of care for this and related diseases.

### SUMMARY

The present disclosure overcomes these and other limitations inherent in the art by providing a multifunctional aptamer-nanomedicine (Apt-NMed) that achieved targeted chemotherapy and gene therapy of ALCL. Apt-NMed was formulated by self-assembly of synthetic oligonucleotides containing CD30-specific aptamer and ALK-specific siRNA followed by self-loading of the chemotherapeutic drug doxorubicin (DOX). Apt-NMed exhibited a well-defined nanostructure (diameter 59 mm) and stability in human serum. Under aptamer guidance, Apt-NMed specifically bound and internalized targeted ALCL cells. Intracellular delivery of Apt-NMed triggered rapid DOX release for targeted ALCL chemotherapy and intracellular delivery of the ALK-specific siRNA induced ALK oncogene silencing, resulting in combined therapeutic effects. Animal model studies revealed that upon systemic administration, Apt-NMed specifically targeted and selectively accumulated in ALCL tumor sites but did not react with off-target tumors in the same xenograft mouse. Importantly, Apt-NMed not only induced significantly higher inhibition in ALCL tumor growth, but also caused fewer or no side effects in treated mice compared to free DOX. Moreover, Apt-NMed treatment markedly improved survival rate of treated mice, opening a new avenue for precision treatment of ALCL.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings form part of the present specification and are included to demonstrate certain aspects of the present invention. For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, and FIG. 1F show the development and characterization of aptamer-nanomedicine. FIG. 1A: Self-assembly scheme of aptamer nanostructure carrying gene specific and cell-specific entities, and the formed Apt-NS/DOX-siRNA (Apt-NMed) inducing targeted chemotherapy and gene therapy. FIG. 1B: Formation of Apt-NS/siRNA was confirmed by change in the size of ssDNA oligonucleotide mixture (ssDNA #1, ssDNA #2, ssDNA #3) on 5% PAGE gel; Lane 1, markers between 50 and 1000 bp; Lane 2, DNA oligonucleotide #1; Lane 3, DNA oligonucleotide #2; Lane 4, DNA oligonucleotide #3; Lane 5, mixture of #1+#2; Lane 6, mixture of #1+#2+#3. FIG. 1C: Formed Apt-NS/siRNA produced two fragments after restriction enzyme digestion with HindIII and BamHI, indicating successful formation of Apt-NS/siRNA; Lane marked with M shows markers between 50 and 1000 bp. FIG. 1D: Saturation point determination of DOX loading in Apt-NS/siRNA by mixing different Apt-NS/siRNA to DOX ratios. Changes in fluorescence were used to monitor DOX intercalation into Apt-NS/siRNA; red arrow indicates saturation point of DOX loading in Apt-NS/siRNA. Size (FIG. 1E) and zeta-potential (FIG. 1F) of Apt-NS/siRNA and Apt-NMed were examined by Zeta-sizer nano-detector;

FIG. 2A, FIG. 2B, FIG. 2C, and FIG. 2D show the morphology and specificity of formed Apt-NMed. Size and morphology characterization of Apt-NMed by AFM (FIG. 2A) and SEM (FIG. 2B). FIG. 2C: SEM imaging clearly displaying Apt-NMed bound to ALCL cells K299, but not to control cells U937. FIG. 2D: Flow cytometry analysis of specific cell binding of Apt-NS and Apt-NS/siRNA to ALCL cells;

FIG. 3A, FIG. 3B, FIG. 3C, FIG. 3D, FIG. 3E, and FIG. 3F show the intracellular delivery of Apt-NMed. FIG. 3A: Determination of internalization pathway of Cy3-labeled-Apt-NS/siRNA by confocal fluorescence microscopy. Upon treatment with endocytosis inhibitor Dynasore, Apt-NS/siRNA (red) internalization was partially blocked in K299 cells, compared to untreated cells. Apt-NS/siRNA uptake was completely blocked in the presence of both endocytosis and macropinocytosis inhibitors. FIG. 3B: Stability of Apt-NMed was determined by incubating with 100% human serum. FIG. 3C: Drug release from Apt-NMed was demonstrated by incubation in PBS buffer along with DNase I or cell lysate. PBS buffer and free DOX were used as negative and positive controls, respectively. Final fluorescence intensity was measured with microplate reader at excitation (470 nm) and emission (590 nm) wavelengths. FIG. 3D: Fluorescence microscopy showed rapid DOX delivery and release into target cells at indicated time points. Flow cytometry analysis further confirmed specific cell binding of Apt-NMed (FIG. 3E) and DOX release (FIG. 3F) in target cells. FAM-labeled Apt-NMed and released DOX were detected with FITC and PE channels of flow cytometer, respectively. Data shown are mean±SEM, n=3; ***p<0.001;

FIG. 4A, FIG. 4B, FIG. 4C, and FIG. 4D illustrate the specific effects of Apt-NMed on ALCL cells for chemotherapy and gene therapy in vitro. FIG. 4A: ALK gene silencing by Apt-NS/siRNA. Cultured Karpas 299 cells were treated with Apt-NS/siRNA for 48 hrs, followed by staining with anti-ALK antibody. Reduction of ALK expression was analyzed by flow cytometry. FIG. 4B: Apt-NS/siRNA induced significant silencing of ALK gene, leading to cell apoptosis and death. FIG. 4C: Fluorescence microscopy imaging of induced-apoptotic and dead cells, detected by AO/EB staining at 24 hrs' post-treatment with Apt-NS/DOX. Viable and dead cells were stained green and red, respectively. FIG. 4D: Rates (%) of apoptotic and dead cells detected by AO/EB staining, at 48 hrs post treatment. Data shown are Mean±SEM, n=3; **p<0.01, ***p<0.001, NS, no statistically-significant difference;

FIG. 5A, FIG. 5B, FIG. 5C, and FIG. 5D illustrate the in vivo targeted delivery of Apt-NS/siRNA into xenograft tumors in mouse model. FIG. 5A: Specific delivery of IRD800 labeled-Apt-NS/siRNA to ALCL xenograft tumors in mouse model detected by IVIS 200 imaging. FIG. 5B: Tumors tissues were removed from mouse, and re-scanned ex vivo. FIG. 5C: Tissue immunostaining with CD30 antibody to confirm that IRD800-Apt-NS/siRNA was delivered into tumors expressing CD30 antibody (magnification ×400). FIG. 5D: Whole body imaging of IRD800-Apt-NS/siRNA versus time to determine particle retention in tumor area;

FIG. 6A, FIG. 6B, FIG. 6C, FIG. 6D, FIG. 6E, and FIG. 6F show the antitumor efficacy of Apt-NMed in vivo. FIG. 6A: Scheme of xenograft tumor treatment: initial treatment took place 9 days post tumor cell inoculation; serial treatment followed by IV administration every other day for four weeks (DOX: 1 mg/kg). FIG. 6B: Bioluminescence imaging of tumor growth in treated and untreated groups (6 mice/group) during Apt-NMed administration. FIG. 6C: Graph showing relative tumor volume change in treated and untreated groups (6 mice/group) post treatment. FIG. 6D: Body weight change of tumor-bearing mice during treatment. FIG. 6E: Kaplan-Meier survival curve of mice bearing ALCL tumors. Mice died naturally or were executed when tumor volume exceeded 2,000 mm³. FIG. 6F Tumor-free percentage of mice (16.7%) post-treatment. Tumor recurrence at 80 days after stopping treatment. Data shown are mean±SEM, n=6; *p<0.05, ***p<0.001;

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and/or time-consuming, but would be a routine undertaking for those of ordinary skill in the art having the benefit of this disclosure.

### Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used throughout this application and in the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

The term “biologically-functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally-equivalent to one or more of the nucleotide sequences provided herein are particularly contemplated to be useful in the practice of the methods and compositions set forth in the instant application.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

The term “for example” or “e.g.,” as used herein, is used merely by way of example, without limitation intended, and should not be construed as referring only those items explicitly enumerated in the specification.

As used herein, a “heterologous” sequence is defined in relation to a predetermined, reference sequence, such as, a polynucleotide or a polypeptide sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, “homologous” means, when referring to polynucleotides, sequences that have the same essential nucleotide sequence, despite arising from different origins. Typically, homologous nucleic acid sequences are derived from closely related genes or organisms possessing one or more substantially similar genomic sequences. By contrast, an “analogous” polynucleotide is one that shares the same function with a polynucleotide from a different species or organism, but may have a significantly different primary nucleotide sequence that encodes one or more proteins or polypeptides that accomplish similar functions or possess similar biological activity. Analogous polynucleotides may often be derived from two or more organisms that are not closely related (e.g., either genetically or phylogenetically).

As used herein, the term “homology” refers to a degree of complementarity between two or more polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

As used herein, “implantable” or “suitable for implantation” means surgically appropriate for insertion into the body of a host, e.g., biocompatible, or having the desired design and physical properties.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed reagents, such as, for example, in a laboratory or clinical application.

“Link” or “join” refers to any method known in the art for functionally connecting one or more proteins, peptides, nucleic acids, or polynucleotides, including, without limitation, recombinant fusion, covalent bonding, disulfide bonding, ionic bonding, hydrogen bonding, electrostatic bonding, and the like.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “operably linked” and operatively linked”, as used herein, refers to that union of the nucleic acid sequences that are linked in such a way, such that the coding regions are contiguous and in correct reading frame. Such sequences are typically contiguous, or substantially contiguous. However, since enhancers generally function when separated from the promoter by several kilobases and intronic sequences may be of variable lengths, some polynucleotide elements may be operably linked but not contiguous.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprines, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. A patient can be of any age at which the patient is able to respond to inoculation with the present vaccine by generating an immune response. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human.

As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, “polymer” means a chemical compound or mixture of compounds formed by polymerization and including repeating structural units. Polymers may be constructed in multiple forms and compositions or combinations of compositions.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures.

For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “1” isomeric form. However, residues in the “d” isomeric form may be substituted for any 1-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “recombinant” indicates that the material (e.g., a polynucleotide or a polypeptide) has been artificially or synthetically (non-naturally) altered by human intervention. The alteration can be performed on the material within or removed from, its natural environment, or native state. Specifically, e.g., a promoter sequence is “recombinant” when it is produced by the expression of a nucleic acid segment engineered by the hand of man. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant AAV virus, is produced by the expression of a recombinant nucleic acid.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment” refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for nucleic acids for use in the present invention include, for example, hybridization in 50% formamide, 5×Denhardt's solution, 5×SSC, 25 mM sodium phosphate, 0.1% SDS and 100 μg/mL of denatured salmon sperm DNA at 42° C. for 16 hr followed by 1 hr sequential washes with 0.1×SSC, 0.1% SDS solution at 60° C. to remove the desired amount of background signal. Lower stringency hybridization conditions for the present invention include, for example, hybridization in 35% formamide, 5×Denhardt's solution, 5×SSC, 25 mM sodium phosphate, 0.1% SDS and 100 μg/mL denatured salmon sperm DNA or E. coli DNA at 42° C. for 16 hr followed by sequential washes with 0.8×SSC, 0.1% SDS at 55° C. Those of ordinary skill in the art will recognize that such hybridization conditions can be readily adjusted to obtain the desired level of stringency for a particular application.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “subject,” as used herein, describes an organism, including mammals such as primates, to which treatment with the compositions according to the present invention can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The terms “substantially corresponds to,” “substantially homologous,” or “substantial identity,” as used herein, denote characteristics of a nucleic acid or an amino acid sequence, wherein a selected nucleic acid sequence or a selected amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

As used herein, “synthetic” shall mean that the material is not of a human or animal origin.

“Targeting moiety” is any factor that may facilitate targeting of a specific site by a particle. For example, the targeting moiety may be a chemical targeting moiety, a physical targeting moiety, a geometrical targeting moiety, or a combination thereof. The chemical targeting moiety may be a chemical group or molecule on a surface of the particle; the physical targeting moiety may be a specific physical property of the particle, such as a surface such or hydrophobicity; the geometrical targeting moiety includes a size and a shape of the particle. Further, the chemical targeting moiety may be a dendrimer, an antibody, an aptamer, which may be a thioaptamer, a ligand, an antibody, or a biomolecule that binds a particular receptor on the targeted site. A physical targeting moiety may be a surface charge. The charge may be introduced during the fabrication of the particle by using a chemical treatment such as a specific wash. For example, immersion of porous silica or oxidized silicon surface into water may lead to an acquisition of a negative charge on the surface. The surface charge may be also provided by an additional layer or by chemical chains, such as polymer chains, on the surface of the particle. For example, polyethylene glycol chains may be a source of a negative charge on the surface. Polyethylene glycol chains may be coated or covalently coupled to the surface using methods known to those of ordinary skill in the art.

The term “therapeutically-practical period” means the period of time that is necessary for one or more active agents to be therapeutically effective. The term “therapeutically-effective” refers to reduction in severity and/or frequency of one or more symptoms, elimination of one or more symptoms and/or underlying cause, prevention of the occurrence of symptoms and/or their underlying cause, and the improvement or a remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally occurring, produced by synthetic or recombinant methods, or a combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and Hardman and Limbird (2001).

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of ordinary skill in the art.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis-sequences that are necessary for efficient transcription and translation (e.g., polyadenylation site(s), mRNA stability controlling sequence(s), etc.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

As used herein, the term “transformed cell” is intended to mean a host cell whose nucleic acid complement has been altered by the introduction of one or more exogenous polynucleotides into that cell.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The tern “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

### Biological Functional Equivalents

Modification and changes may be made in the structure of the nucleic acids, or to the vectors comprising them, as well as to mRNAs, polypeptides, or therapeutic agents encoded by them and still obtain functional systems that contain one or more therapeutic agents with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions or corresponding DNA sequences which encode said peptides without appreciable loss of their biological utility or activity.

TABLE 1

| AMINO ACIDS | | CODONS | | | | | |
|---|---|---|---|---|---|---|---|
| Alanine | Ala | GCA | GCC | GCG | GCU | | |
| Cysteine | Cys | UGC | UGU | | | | |
| Aspartic acid | Asp | GAC | GAU | | | | |
| Glutamic acid | Glu | GAA | GAG | | | | |
| Phenylalanine | Phe | UUC | UUU | | | | |
| Glycine | Gly | GGA | GGC | GGG | GGU | | |
| Histidine | His | CAC | CAU | | | | |
| Isoleucine | Ile | AUA | AUC | AUU | | | |
| Lysine | Lys | AAA | AAG | | | | |
| Leucine | Leu | UUA | UUG | CUA | CUC | CUG | CUU |
| Methionine | Met | AUG | | | | | |
| Asparagine | Asn | AAC | AAU | | | | |
| Proline | Pro | CCA | CCC | CCG | CCU | | |
| Glutamine | Gln | CAA | CAG | | | | |
| Arginine | Arg | AGA | AGG | CGA | CGC | CGG | CGU |
| Serine | Ser | AGC | AGU | UCA | UCC | UCG | UCU |
| Threonine | Thr | ACA | ACC | ACG | ACU | | |
| Valine | Val | GUA | GUC | GUG | GUU | | |
| Tryptophan | Trp | UGG | | | | | |
| Tyrosine | Tyr | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporate herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index based on its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively based on hydrophilicity. U.S. Pat. No. 4,554,101 (specifically incorporated herein in its entirety by express reference thereto), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take one or more of the foregoing characteristics into consideration are well known to those of ordinary skill in the art, and include arginine and lysine; glutamate and aspartate; serine and threonine; glutamine and asparagine; and valine, leucine and isoleucine.

The section headings used throughout are for organizational purposes only and are not to be construed as limiting the subject matter described. All documents, or portions of documents, cited in this application (including, but not limited to, patents, patent applications, articles, books, and treatises) are expressly incorporated herein in their entirety by express reference thereto. In the event that one or more of the incorporated literature and similar materials defines a term in a manner that contradicts the definition of that term in this application, this application controls.

### Nanomedicines

Nanomedicines offer notable advantages compared to traditional drug delivery methods including transportation of higher drug payloads, prolonged drug circulation and improved bioavailability, synergistic effects due to simultaneous administration of multiple drugs with different activities, and improved therapeutic indices through enhanced permeability, retention, and programmed design (Tigli-Aydin, 2015; Auffan et al., 2009; Toy et al., 2014).

Recent studies showed that single-stranded DNA nanostructures provide tunable, precise, rationally designed drug delivery vehicles (Chen et al., 2015; Angell et al., 2016; Jiang et al., 2016) that can be further functionalized with judiciously chosen ligands for cell-specific and targeted therapy (Lee et al., 2012; Liu et al., 2016; Zhu et al., 2014; (Zhu et al., 2013).

### Aptamers

Aptamers are a class of short, single-stranded oligonucleotides (RNA or ssDNA) with high affinity and specificity for a wide range of biological targets (Banerjee et al., 2013; Wandtke et al., 2015; Zimbres et al., 2013; Deng et al., 2014).

Similar, to protein antibodies, aptamers bind to their cognate targets via conformational recognition and thus, are often referred to as “chemical antibodies.” As small-sized oligonucleotide biomaterials, aptamers can be rapidly synthesized and easily conjugated to various functional agents for different clinical needs (Jo et al., 2016; Chen et al., 2016; Gopinath et al., 2016).

The aforementioned features provide aptamers with notable advantages over protein antibodies as targeting ligands to formulate nanoparticles and nanomedicines (Sun et al., 2016; McKeague and Derosa, 2012; Friedman et al., 2013; Levy-Nissenbaum et al., 2008).

### T-Cell Lymphoma

Anaplastic large cell lymphoma (ALCL) is the most common T-cell lymphoma in children. Biologically, lymphoma cells express high-levels of the surface CD30 receptor, a signaling molecule regulating cell fate and a distinct biomarker for ALCL diagnosis. Genetically, lymphoma cells express the Anaplastic Lymphoma Kinase (ALK) oncogene, a key pathogenic factor for lymphoma development in the majority of children. Currently, CHOP chemotherapy, comprising cyclophosphamide, hydroxyl-doxorubicin, oncovin (Vincristine) and prednisone, is the mainstay of treatment for ALK+ALCL (Ma et al., 2017; Fisher et al., 1993). However, the CHOP regimen is not ALCL cell-selective or ALK gene-specific, and thus, has serious adverse side effects. Recently, FDA approved CD30 antibody drug-conjugates for cell-targeted chemotherapy treatment of relapsed/refractory ALCL (de Goeij et al., 2016; Diefenbach and Leonard, 2012). Also, RNA interference (RNAi) approaches (Hsu et al., 2007; Zhao et al., 2011) and kinase inhibitors (Turturro et al., 2002; Galkin et al., 2007) to restrain expression or activity of ALK oncogene have been investigated. Moreover, stimulation of CD30 by induced polymerization led to apoptotic death of ALK+ALCL cells in vitro and antitumor effects in animal model (Schirrmann et al., 2014; Pierce et al., 2017). However, treatment with single therapeutic modalities appears to be ineffective in curing ALK+ALCL, and also carries the risk of inducing drug-resistance of lymphoma cells.

### Example

The following example is included to demonstrate illustrative embodiments of the invention. It should be appreciated by those of ordinary skill in the art that the techniques disclosed in this example represents techniques discovered to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### Preparation of Self-Assembled Aptamer-Nanomedicine Compositions

To address the aforementioned clinical challenges, we previously reported a functionalized siRNA nanocomplex, consisting of a CD30-specific aptamer and ALK gene-specific siRNA on nanosized polyethylenimine (PEI) polymer carriers (Zhao et al., 2011). Functional analysis revealed that the siRNA-aptamer nanocomplexes preferentially bound to and were internalized by ALCL cells, whereas they did not bind to off-target cells, resulting in down-regulation of cellular ALK gene expression and growth inhibition of ALCL cells. Notwithstanding the advantages of the functionalized nanocomplex for targeted ALCL therapy, its further development for clinical use is limited due to the unknown risks of PEI as a foreign material to the human body and the short lifetime of RNA-based aptamers due to susceptibility to environmental nucleases.

To overcome the shortcomings of the previous nanocomplex (Zhao et al., 2011), in this example, a multifunctional aptamer-nanomedicine (Apt-NMed) is described that is useful in ALCL therapy, through self-assembly of functional oligonucleotides, comprising aptamers and siRNA sequences, followed by self-loading of the therapeutic drug doxorubicin (DOX). As synthetic biodegradable oligonucleotides, the DNA-based aptamers rendered Apt-NMed both biostable and biocompatible. Functional studies demonstrated that Apt-NMed achieved both ALCL cell-targeted chemotherapy and ALK gene-specific therapy. The therapeutic efficacy of Apt-NMed was validated in a preclinical model. Studies showed that the resulting combination therapy not only achieved higher therapeutic efficacy but also had fewer non-specific toxic side effects on normal cells/tissues.

It is shown herein that Apt-NMed, which can be simply formulated through self-assembly of functional oligonucleotides containing and self-loading of the chemotherapeutic drug DOX, successfully performed selective chemotherapy and gene therapy of ALCL. Under CD30-specific aptamer guidance, the multifunctional Apt-NMed specifically bound ALCL cells and performed targeted chemotherapy through intracellular delivery of DOX and specific oncogene silencing via intracellular delivery of ALK-specific siRNA. The combination of cell-selective chemotherapy and gene therapy of Apt-NMed achieved higher growth inhibition rates of ALCL tumors and significantly higher survival rates of xenograft mice as compared to single agent therapy. Importantly, treatment with Apt-NMed showed negligible toxicity in mice in contrast to treatment with an equimolar amount of free DOX, which caused severe toxicity in mice.

Notably, as a synthetic biomaterial, Apt-NMed is completely biocompatible and biodegradable, and will not cause adverse effects in the human body. The majority of materials previously conjugated to aptamers, such as inorganic and organic carriers (Sun and Zu, 2015) including gold or magnetic nanomaterials (Zhao et al., 2013; Pala et al., 2014; Wang et al., 2013; Shiao et al., 2014), single-walled carbon nanotubes (Taghdisi et al., 2011, de Puig et al., 2013, Zhu et al., 2008) mesoporous silica nanoparticles (He et al., 2012; Zhu et al., 2011), quantum dots, liposomes, copolymers, and protein-based nanomaterials (Farokhzad et al., 2006; Xing et al., 2013; Zhang et al., 2007; Huang et al., 2014; Li et al., 2014; Wu et al., 2013) are foreign to the human body and may pose unknown risks for in vivo use. It is noteworthy that Apt-NMed can be synthesized by a simple method, which is highly reproducible, cost-effective, and can be easily scaled up for industrial production of this multifunctional nanomedicine. Apt-NMed can also be developed to logically combine multiple therapeutic modalities for additional or synergistic efficacy, including chemotherapy, gene therapy (Li et al., 2014; Guo et al., 2005; Shu et al., 2011; Haque et al., 2012; Zhou et al., 2011), immunotherapy, biotherapy, kinase inhibitors, thermotherapy (Pala et al., 2014; Wang et al., 2013), and photodynamic therapy (Wang et al., 2013; Shiao et al., 2014). The design of Apt-NMed is contingent upon several factors aimed at precision therapy, such as the nature of the disease, biology of cells/tissues of interest, chemical and physical properties of aptamers, selection of appropriate nanomaterials, and appropriate therapeutic agents/drugs. The aforementioned findings provide a solid foundation for development of new precision nanomedicines that can selectively treat ALCL and other diseases with high efficacy and minimal or no adverse effects in patients. Further preclinical and clinical studies are necessary to translate Apt-NMed from bench to bedside.

### Materials and Methods

### Reagents and Cell Lines

Karpas299 (K299) cells expressing receptor CD30 were used in a collaboration at the National Cancer Institute/National Institutes of Health. CD30-negative U937 and MDA-MB-231 cells were purchased from the American Type Culture Collection (ATCC, Manassas, Va., USA). K299 and MDA-MB231 cells, stably expressing GFP and luciferase, were used for tumor formation in mouse model. Suspension cells K299 and U937 were cultured with RPMI1640 medium (Fisher Scientific, Pittsburgh, Pa., USA) with 10% FBS (Atlanta Biologicals, Lawrenceville, Ga., USA). Adhesion cells MDA-MB-231 were cultured in DMEM (Atlanta Biologicals, Lawrenceville, Ga., USA) with 10% FBS. The chemotherapeutic drug DOX was purchased from Sigma (St. Louis, Mo., USA).

### Synthetic Oligonucleotides and Formation of Aptamer Nanostructure

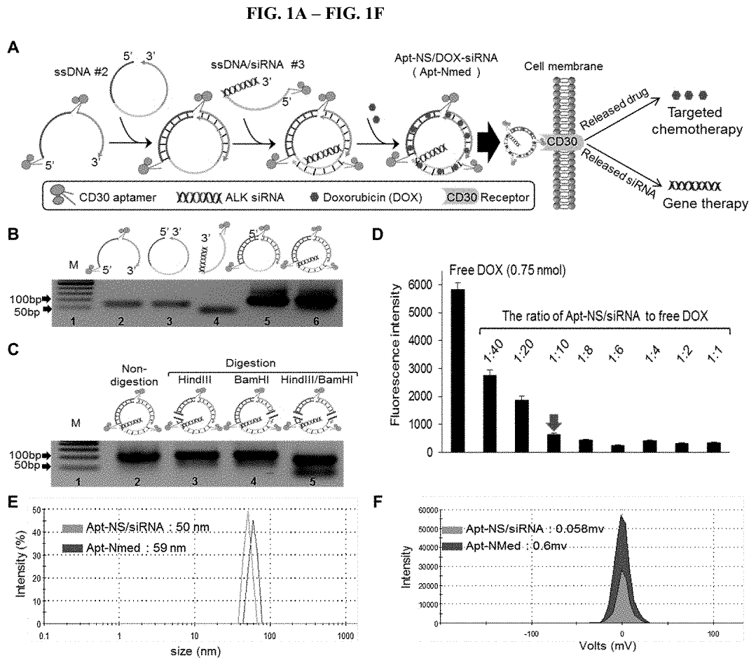

To generate the aptamer nanostructure linked with siRNA (Apt-NS/siRNA), three single-stranded oligonucleotides containing aptamer, complementary ssDNA and/or siRNA sequences were synthesized. To ensure biosafety, synthetic complementary sequences were adapted from human IgG heavy chain (NCBI database: GenBank: X82593.1). To target cells of interest, ssDNA CD30-specific aptamer was employed as previously reported (Parekh et al., 2013). For oncogene silencing, the previously validated ALK-specific siRNA sequence (Zhao et al., 2011) was directly linked to the aptamer. To validate aptamer-nanostructure formation, two endonuclease restriction sites, namely HindIII and BamHI (underlined nucleotides in sequences shown below), were introduced into the arm sequences of ssDNA. All oligonucleotides were synthesized by Integrated DNA Technologies (IDT, Coralville, Iowa, USA). The scheme for self-assembly of the aptamer nanostructure is depicted in FIG. 1A. In addition, Apt-NS without siRNA, and control NS or NS/siRNA without aptamer were also synthesized. The ssDNA sequence for aptamer nanostructure formation is displayed below:

ssDNA #1: CD30 aptamer/ssDNA/CD30 aptamer/ssDNA
5′ ACTGGGCGAAACAAGTCTATTGACTATGAGCGGTAAAGCTTGTGTAG
AGAGaaaACTGGGCGAAACAAGTCTATTGACTATGAGCaaaTGGATCCAG
GACTAGTAAAGC 3′
ssDNA #2: ssDNA/ssDNA/ssDNA
5′ CTCTCTACACAAGCTTTACCaaAAAAAAAAAAAAAAAAATTaaGCTT
TACTAGTCCTGGATCCA 3′
ssDNA/siRNA #3: CD30 aptamer/ssDNA/siRNA
5′ ACTGGGCGAAACAAGTCTATTGACTATGAGCaaAATTTTTTTTTTTT
TTTTTaaCACUUAGUAGUGUACCGCC 3′
Antisense anti-ALK siRNA:
5′ GGCGGUACACUACUAAGUG 3′

Apt-NS/siRNA was formed by self-assembly of three single-stranded oligonucleotides via programmed hybridization of complementary ssDNA sequences. Briefly, synthetic oligonucleotides were mixed in equal molar ratio (1:1:1), each at 10 μM final concentration, in TRIS EDTA buffer (Sigma Aldrich, St. Louis, Mo., USA) supplemented with 10 mM MgCl₂ (Fisher Scientific, Pittsburgh, Pa., USA). Reactions were heated at 95° C. for 5 min and then cooled to 4° C. for programmed hybridization of oligonucleotides to form Apt-NS/siRNA.

### Characterization of Apt-NS/siRNA Formation by Native PAGE

First, the formed Apt-NS/siRNA (1 μg) was separated on 5% non-denaturing PAGE and analyzed on a Gel Doc EZ imager (Bio-Rad, Hercules, Calif., USA). The individual oligonucleotides (ssDNA #1, ssDNA #2, and ssDNA/siRNA #3), and a mixture of oligonucleotides (ssDNA #1+ssDNA #2) were treated under the same conditions as the controls (FIG. 1B). For further confirmation, the annealed aptamer nanostructure (1 μg) was digested with the restriction endonucleases HindIII and/or BamHI for at least 2 hrs, in an incubator at 37° C., and analyzed by PAGE (FIG. 1C).

Drug Loading into Apt-Ns/siRNA

DOX intercalation can be measured by monitoring changes in DOX fluorescence because DOX fluorescence is dramatically quenched upon intercalation into DNA. Briefly, to determine the loading ability of Apt-NS/siRNA for DOX, Apt-NS/siRNA and DOX were mixed at specific molar ratios (from 1:1 to 1:40) and kept overnight at room temperature to allow saturation of drug loading (DOX was fixed to 0.1 nmol). Fluorescence intensity was measured by microplate reader (Eₑₓ=470 nm, Eₑm=590 nm, Biotek, Winooski, Vt., USA). Saturation of DOX loading was determined based on fluorescence intensity of free DOX.

### Dynamic Light Scattering Analysis of Apt-NS/siRNA and Apt-NMed

The size and zeta-potential of the formed Apt-NS/siRNA and drug-loaded Apt-NS/siRNA (10 nM) were characterized with a Zetasizer Nano ZS ZEN3600 (Malvern Instruments, Worcestershire, UNITED KINGDOM); the scattering angle was 173°. Three sets of measurements, each comprising 15 runs with a 20 sec duration for each, were performed at 25° C. For the calculations, a viscosity, of 0.8872 mPa sec, and a refractive index of 1.33 were used. The autocorrelation function was evaluated by ALV-Correlation software v.3.0, using an exponential regularized fit to get the size distribution curves and Zetasizer Software 6.34 to get the z-average and PDI values.

### Characterization of Apt-NMed

Samples were studied by AFM and SEM to determine the size and morphology of freshly made Apt-NMed. For AFM imaging, prior to sample loading, the sample holder mica was pre-treated with 0.1% vol/vol) APTES (3-aminopropyltriethoxy-silane) water solution for 10 min, washed with pure water, and then dried with compressed nitrogen. The formed nanostructure Apt-NMed was diluted to 10 nM in Tris buffer supplemented with 10 mM MgCl₂ in 10 μL; this solution was placed on a previously modified surface, absorbed for 5 min, washed with pure water, and dried with compressed nitrogen for imaging with MultiMode M8 AFM (Bruker, Spring, Tex., USA). For high-resolution images, an SNL-10 probe (Veeco, Inc., USA), with super-sharp tips (2-3 nm radius), was used. For SEM imaging, a drop of solution (5 μL, 10 nM) was spotted onto silicon pre-treated with aminopropylsilane (APS) for 5 min to allow strong adsorption. The samples were air-dried and carefully mounted on an aluminum stub using either silver paint or a double-stick carbon tape. Samples were then placed in the chamber of the sputter coater, and coated with a very thin film of gold. All SEM images were taken at 10-15 kV (accelerating voltage) with electron-beam spot size 3, in the FEI Nova NanoSEM 230 (Hillsboro, Oreg., USA). Chamber pressure was between 1.0×10⁻⁷ and 1.0×10⁻⁶ mbar (1 mbar=100 Pa).

### Specific Cell Binding of Apt-NMed to ALCL Cells Analyzed by SEM

Briefly, the treated cells were fixed with 2.5% glutaraldehyde at 4° C. for 2 hrs, followed by sequential dehydration for 10 min in 20, 30, 50, 70, 90, and 100% ethanol. The fixed cells were sputter-coated with PtPd using a 208 HR Sputter Coater (Cressington Scientific, Cranberry Twp, Pa., USA), and examined by SEM (Nova NanoSEM 230, FEI Co., Hillsboro, USA).

### Specific Cell Binding of Apt-NS and Apt-NS/siRNA to ALCL Cells Analyzed by Flow Cytometry

Cy3-labeled aptamer nanostructure (Apt-NS), control Cy3-labeled control nanostructure (NS), Cy3-directly labeled CD30 aptamer (Cy3-Apt) and CD30 antibody were incubated with 1×10⁵ K299 and U937 cells at 100 nM for 30 min at room temperature (RT). The cells were washed once with 500 μL PBS, and re-suspended in 500 μL PBS for flow cytometry analysis. Fluorescence intensity was determined with a FACScan cytometer (LSRII, BD Biosciences, San Jose, Calif., USA) by counting 10,000 events.

### Internalization Pathway of Apt-NS/siRNA

To examine internalization pathway of Apt-NS/siRNA, K299 cells were incubated with both Cy3-labeled Apt-NS/siRNA (100 nM) and endocytosis inhibitor Dynasore (80 μM) or Cy3-labeled Apt-NS/siRNA along with endocytosis inhibitor Dynasore (80 μM) and micropinocytosis inhibitor Cytochalasin D (1 μM) for 2 hrs at 37° C.; Cy3-labeled Apt-NS/siRNA (100 nM) alone was used as control. Post-PBS buffer washing, the fixed cells were visualized by confocal fluorescence microscopy (Olympus, FluoView® 1000).

### Stability of Apt-NMed

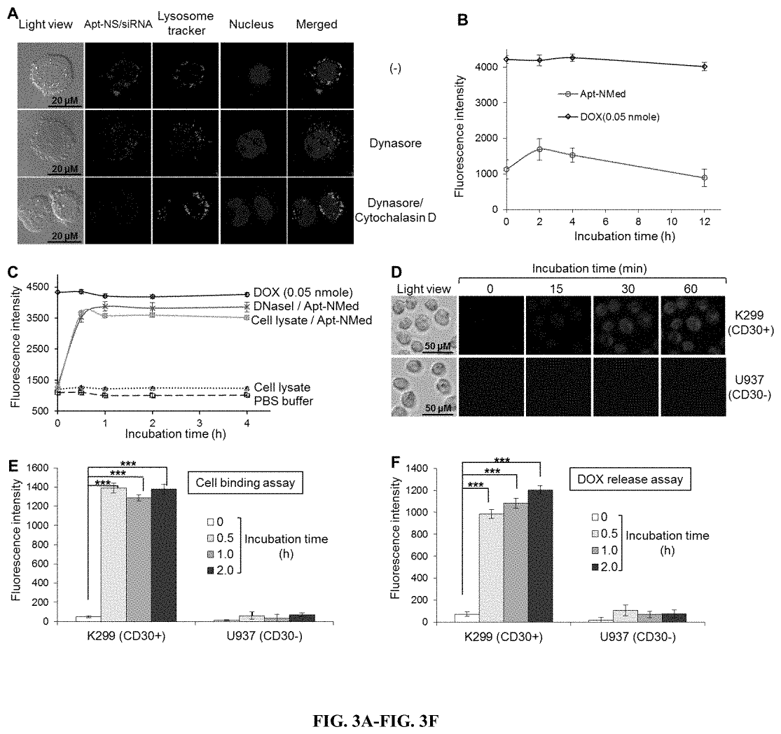

To detect stability of nanostructure/DOX conjugates, Apt-NMed (100 nM) was incubated at 37° C. in 100 μL of 100% human serum (Atlanta Biological, Lawrenceville, Ga., USA). At the times indicated in FIG. 3B, fluorescence intensity of the released DOX was detected by microplate reader, at the excitation and emission wavelengths 480±20 and 570±20 nm, respectively (Biotek, Winooski, Vt., USA).

Drug Release from Apt-NMed by DNA Degradation

The formed Apt-NMed (100 nM; ratio of nanostructure to DOX=1:10) was incubated at 37° C. in 100 μL of PBS buffer with 2 units of DNase I (Life technologies, Grand Island, N.Y., USA), or cell lysate from K299 cells for 2 hrs. Fluorescence from the released DOX was detected at 590 nm after excitation at 480 nm (Biotek, Winooski, Vt., USA). Cell lysate and PBS buffer alone were considered negative controls. The same amounts of free DOX were considered as controls for 100% drug release from Apt-NMed.

### Intracellular Delivery of Apt-NMed Observed by Fluorescence Microscopy and Flow Cytometry

Apt-NMed (100 nM) was incubated with K299 and U937 cells at 37° C. Fluorescent signal from the released DOX was recorded by a fluorescence microscope (Olympus FluoView® 1000, Olympus America, and Center Valley, Pa., USA) at different time points (FIG. 3D). Cell-selective intracellular delivery and drug release from Apt-NMed were also determined by flow cytometry (BD Biosciences, San Jose, Calif., USA). Briefly, DOX was loaded into FAM-labeled Apt-NMed to treat K299 and U937 cells. As illustrated in FIG. 3D, cells were collected, and analyzed by flow cytometry at different time points. Cell binding to Apt-NMed and released DOX were detected using FITC and PE channels, respectively. The efficiency of particle delivery and drug release was determined by quantification of changes in fluorescence.

### Silencing of ALK Gene by Apt-NS/siRNA

To address of silencing of ALK expression by Apt-NS/siRNA, cultured Karpas 299 cells were treated with 100 nM Apt-NS containing ALK-targeted siRNA, or 100 nM siRNA for 4 hrs at 37° C. The previous solution was removed; cells were treated with fresh medium (10% FBS), and cultured for 24 hrs. Cells were collected and stained with FITC-labeled mouse anti-human ALK antibody (BD Biosciences, San Jose, Calif., USA), then analyzed by flow cytometry to determine silencing of ALK (BD Biosciences, San Jose, Calif., USA). To determine the effect of ALK gene knockdown on cell growth and the rates of apoptotic (very weak green) and dead cells (bright red) in the treated and control groups, the collected cells were stained with AO (5 μg/mL)/EB (3 μg/mL) and analyzed by flow cytometry.

### Demonstration of Specific and Enhanced Effect of Apt-NS/DOX on Target Cells by Fluorescence Microscopy

Samples containing 25 nM Apt-NS/DOX (DOX 0.25 μM) and free DOX (0.25 μM) were incubated with K299 (CD30⁺) cells and U937 (CD30⁻) at RT for 2 to 4 hrs, respectively. The treatment solution was removed, and complete fresh medium was added to continue culturing for 24 hrs. The treated cells were stained with AO (5 μg/mL)/EB (3 μg/mL), according to the manufacturer's instructions. After washing, slides of cell smears were prepared and examined under a fluorescence microscope (Olympus FluoView® 1000, Olympus America, Center Valley, Pa., USA). Viable and dead cells were stained green and red, respectively.

### Combined Effect of Apt-NMed on ALCL Cells

To determine the combined effect of Apt-NMed, K299 cells were treated with aptamer nanostructures including Apt-NS, Apt-NS/siRNA, Apt-NS/DOX, Apt-NMed, and control nanostructures including NS, NS/siRNA, NS/DOX, NS/DOX-siRNA, in RPMI 1640 medium for 2 hrs. After replacement with fresh medium containing 10% serum, cells were cultured for an additional 48 hrs, stained with AO (5 μg/mL)/EB (3 μg/mL) for 5 min at RT, and analyzed by flow cytometry to determine the apoptosis (very weak green) and death (bright red) rates in the treated and control groups.

In Vivo Targeted Delivery of Apt-NS/siRNA into Xenograft Tumors in A Mouse Model

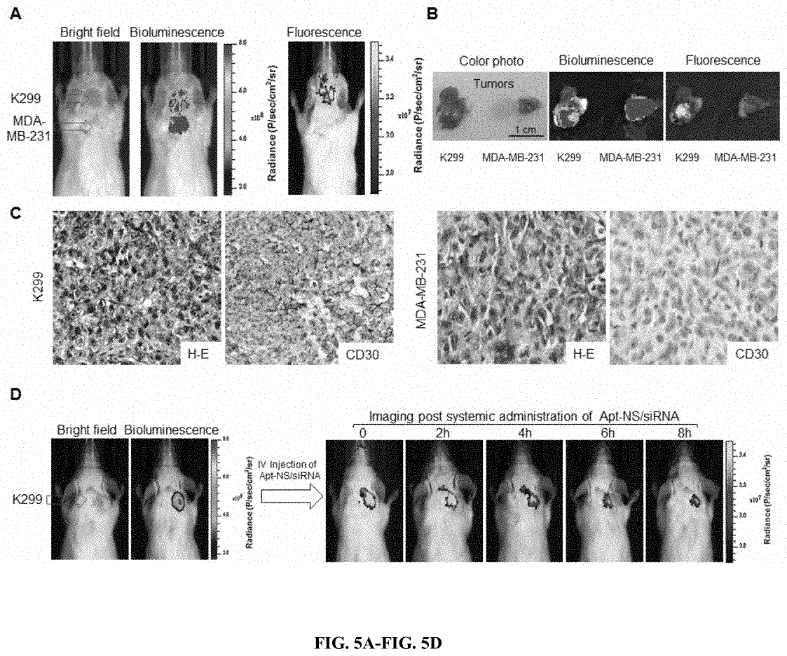

For imaging studies, 4-6-weeks old NOD.Cg-Prkdc^{scid} Il2rg^{tm1Wj1}/SzJ (NOD SCID) mice were purchased from Jackson Laboratory (Bar Harbor, Me., USA). Each mouse was subcutaneously inoculated with cultured CD30-expressing lymphoma cells (Karpas 299, 6×10⁶) and CD30-negative control tumor cells (MDA-MB-231; 2×10⁶) at the same time, but at different anatomic sites, as shown in FIG. 5A. Both cells stably expressed Green fluorescence protein (GFP) and luciferase. Tumor development was monitored by bioluminescence scanning and confirmed by physical examination until the tumor reached a diameter ≥5 mm (approximately 20-25 days post-tumor cell implantation). Apt-NS/siRNA labeled with reporter IRD800CW (6.7 μg in 100 μL PBS buffer) was administered systemically via tail vein injection, and whole-body imaging using the IVIS 200 Imaging System was performed immediately and up to 6 hrs after injection, as shown in FIG. 5D. Imaging signals of regions of interest (ROIs) from lymphoma tumors, control tumors and body areas without tumor were recorded in digital format, using Xenogen software. The increase in signal intensity was calculated according to the manufacturer's instructions as follows:

$$Fold increase = \frac{{Tumor}_{correct} dye - {Tumor}_{correct} none )}{( {Blank}_{correct} \_dye - {Blank}_{correct} \_none )}$$

To confirm-imaging findings, tumors and adjacent tissues were removed at the end-point and re-scanned as previously described. Cells extracted from tumor tissues were observed by fluorescence microscopy. In addition, tumor tissues were fixed and immunostained for CD30 expression in our pathology laboratory, following a standard protocol.

### Anticancer Efficacy of Apt-NMed In Vivo

Mice NOD.Cg-Prkdc^{scid} Il2rg^{tmlWjl}/SzJ (NOD SCID), 4-6-weeks old, were used to develop xenograft tumor models by subcutaneously injecting 6×10⁶ cultured CD30-expressing lymphoma cells (Karpas 299) (in 100 μL of PBS buffer) into mice on the back. Dorsal tumor nodules were allowed to grow to a volume of ˜4 mm³ before treatment initiation. Tumor-bearing mice were randomly assigned to three groups (with six mice in each group) as follows: (i) group administered with 0.25 mg Apt-NMed carrying 20 DOX to each mouse; (ii) group administered 20 μg free DOX to each mouse, and (iii) untreated group. Drugs were injected through tail veins every other day, and the treatment was continued for four weeks. Tumor length and width were measured with calipers weekly for each mouse. Tumor volume was calculated using the following equation:

Tumor Volume=Length×Width²×0.52

The body weight of each mouse was also measured weekly to monitor potential drug toxicity. Mice were euthanized when tumor volume exceeded 2,000 mm³.

### Results and Discussion

### Development and Characterization of Aptamer-Nanomedicine

Apt-NMed was simply formulated through self-assembly of functional oligonucleotide sequences and self-loading of DOX (FIG. 1A). It was initially hypothesized that the Apt-NMed would specifically bind and act on ALCL cells under CD30-specific aptamer guidance. It would then induce combined cell-targeted chemotherapy—via intracellular release of its drug payload—and cell-targeted gene therapy to achieve ALK oncogene silencing via intracellular delivery of siRNA.

To develop Apt-NMed, an aptamer-nanostructure carrying siRNA (Apt-NS/siRNA) was initially formulated through a self-assembly process (FIG. 1A). To this end, three complementary functional single-stranded DNA (ssDNA) sequences were synthesized, including CD30-specific aptamer sequences to target ALCL cells (Parekh et al., 2013) and ALK-specific siRNA for oncogene silencing (Hsu et al., 2007; Zhao et al., 2011). As evident in the studies above, the synthetic oligonucleotide ssDNA #1 contains two separate CD30 aptamers and two complementary arm sequences; ssDNA #2 contains three complementary arm sequences for nanostructure formation; and ssDNA/siNRA #3 contains a complementary arm with an aptamer sequence at 5′-end and a chimeric siRNA sequence at 3′-end. Notably, for biosafety purposes, the complementary arm sequences were derived from human immunoglobulin sequences. For the purpose of validation, restriction sites for two endonucleases were introduced into the arm sequences of the Apt-NS/siRNA, as described below. Additional nanostructures were also prepared and used in the control experiments. The Apt-NS/siRNA was formed by simply mixing equimolar amounts of three synthetic complementary oligonucleotides, and self-assembly was confirmed by polyacrylamide gel electrophoresis (PAGE). Compared to the individual ssDNA oligonucleotides, which had different mobility shifts on PAGE gel, the Apt-NS/siRNA products generated a single band, indicating nanostructure formation (FIG. 1B). In addition, dynamic light scattering (DLS) analysis showed that the three assembled ssDNA sequences formed the compact particle Apt-NS/siRNA with a single peak at 50 nm, whereas the parental individual ssDNA sequences had peaks ranging from 80 to 250 nm. For further validation, Apt-NS/siRNA was exposed to restriction endonucleases, and the resultant products were analyzed by PAGE. As shown in FIG. 1C, two digested fragments were detected post-treatment with endonucleases HindIII and BamHI. The previous findings confirmed Apt-NS/siRNA formation through self-assembly of three complementary ssDNA sequences because endonucleases only cleave dsDNA nanostructures.

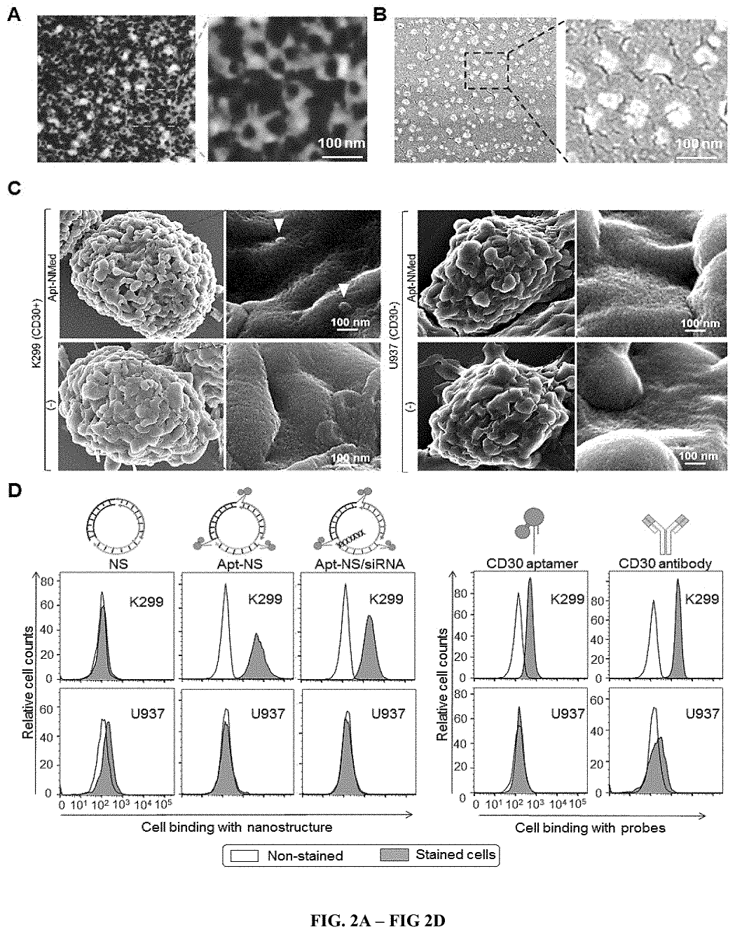

Subsequently, self-loading of DOX in Apt-NS/siRNA was conducted given that DOX is a key component of the chemotherapeutic regimen for ALCL. Notably, due to its chemical properties, DOX can freely intercalate into dsDNA (GC or CG sequences) via non-covalent interactions (Porciani et al., 2015; Perez-Arnaiz et al., 2014). Therefore, the Apt-NS/siRNA was incubated with free DOX overnight, at different molar ratios ranging from 1:1 to 1:40 (FIG. 1D). Given that fluorescence of free DOX is quenched upon intercalation into dsDNA (Agudelo et al., 2014; Zunino et al., 1980), self-loading of DOX into Apt-NS/siRNA was quantified by detecting fluorescence of residual free DOX using a microplate reader (Eₑₘ=590 nm). As shown in FIG. 1D, the Apt-NS/siRNA fully incorporated up to 10-fold DOX payload (mol./mol.). The physical properties of the formulated Apt-NS/DOX-siRNA (Apt-NMed) were then characterized. DLS assay indicated that DOX self-loading had minimal effect on Apt-NMed size because it had a peak at 59 nm (FIG. 1E). In addition, the zeta-potentials of the aptamer-nanomedicine became more positive than that of Apt-NS/siRNA (0.60 vs. 0.058) (FIG. 1F), indicating that Apt-NMed is more stable than aptamer nanostructure. Furthermore, atomic force microscopy (AFM) and scanning electron microscopy (SEM) studies were performed (FIG. 2A and FIG. 2B) and revealed the uniform size and shape of Apt-NMed, in accordance with the findings from measurements of the physical properties.

### Apt-NMed Specifically Targeted ALCL Cells for Both Chemotherapy and Gene Therapy

To examine cell binding, cultured cells were treated with Apt-NMed and examined by SEM. The Apt-NMed specifically bound ALCL cells (K299), but did not react with control U937 cells, which do not express surface CD30 (FIG. 2C). In addition, individual nanostructures were labeled with fluorochrome Cy3 reporter and incubated with cultured cells. Flow cytometry analysis revealed that Apt-NS and Apt-NS/siRNA specifically bound to K299 cells, but not to U937 cells, with similar reaction patterns to those observed with CD30 aptamer or anti-CD30 antibody (FIG. 2D). However, the control nanostructure (NS) alone showed no cell binding due to lack of conjugated aptamer.

Recent studies showed that specific cell binding of aptamers resulted in endocytosis into lysosomes and macropinocytosis into cytoplasm (Zhou and Rossi, 2011; Meyer et al., 2011). To validate the potential mechanism of intracellular delivery, the Apt-NS/siRNA was labeled with fluorochrome Cy3 and incubated with K299 cells, in which lysosomes had been pre-stained with LysoTracker Green for tracking purposes. Confocal microscopy studies revealed intracellular delivery of Apt-NS-siRNA in the cytoplasm only (red fluorescence) and co-localization with lysosomes (merged yellow signal from red and green) as shown in FIG. 3A. To distinguish between the intracellular delivery pathways, K299 cells were pre-exposed to an endocytosis inhibitor (Dynasore). Blockage of endocytosis eliminated lysosomal delivery of Apt-NS/siRNA, but had no effect on cytoplasmic delivery. Moreover, pre-exposure of K299 cells to both endocytosis and macropinocytosis (Cytochalasin D) inhibitors completely abolished intracellular delivery of Apt-NS/siRNA, confirming presence of two different intracellular delivery pathways, namely endocytosis to cell lysosomes and macropinocytosis to cytoplasm.

An ideal delivery system for targeted therapy should be stable under physiological conditions and also have the ability to release drug payload in cells of interest. Therefore, the Apt-NMed was incubated in human serum and its stability was evaluated by kinetically detecting released DOX given that intercalated DOX is optically silent. Fluorescence measurements with a microplate reader revealed that Apt-NMed was stable in serum and release of free DOX was not detected post incubation for 12 hrs (FIG. 3B). Apt-NMed was also stable in fresh plasma from human blood or under different pH values ranging from 4.0 to 7.4. To test drug release potential, Apt-NMed was treated with DNase I or fresh ALCL cell lysates as a source of cellular nucleases. Resultant degradation of Apt-NMed led to rapid release of DOX payload, reaching the maximum within 30 min (FIG. 3C). To confirm intracellular drug release capacity, cultured cells were treated with Apt-NMed and examined under the fluorescence microscope. Intracellular fluorescence, derived from free DOX that was released from the degraded Apt-NMed was detected in K299 cells 15 min post treatment, and reached the maximum at 1 hr (FIG. 3D). For further validation, the Apt-NMed was labeled with FITC fluorescent reporter and incubated with cultured cells. Flow cytometry analysis showed that Apt-NMed specifically bound to K299 cells and reached maximal binding in 30 min, but did not react with U937 cells (FIG. 3E). Quantitative flow cytometry analysis detected intracellular DOX release in K299 cells with maximal release in 2 hrs, but no DOX in control U937 cells (FIG. 3F). Taken together, these findings indicate that Apt-NMed specifically targeted K299 cells, was internalized in nuclease-rich lysosomes via endocytosis, and thus, released drug payload exclusively within cells of interest.

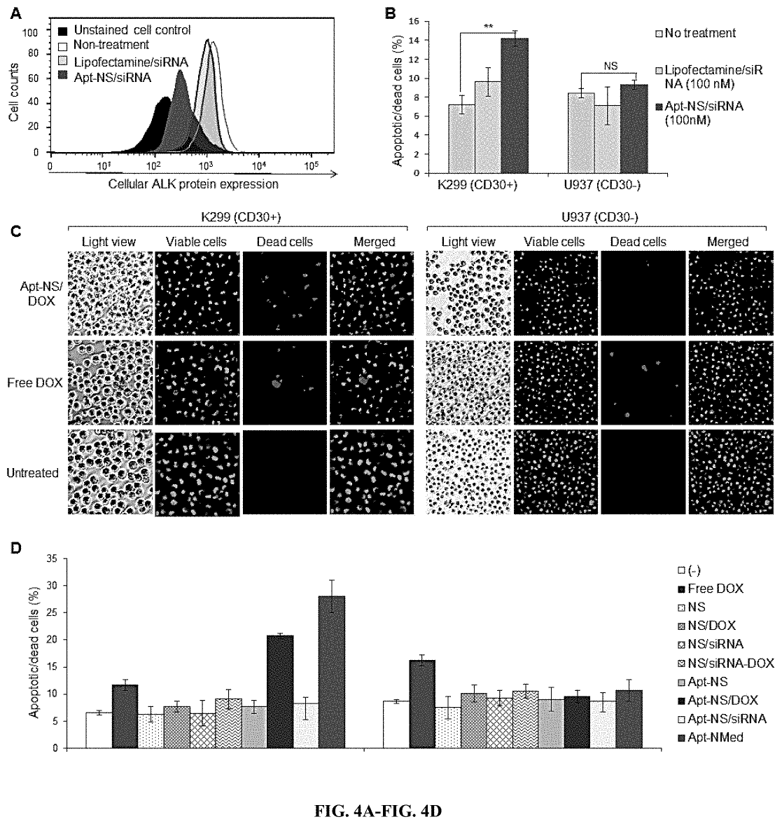

On the other hand, specific cell binding of Apt-NS/siRNA also triggered cytoplasmic delivery via macropinocytosis (FIG. 3A), which has been demonstrated to allow siRNA-induced gene silencing (Hou et al., 2013; Reyes-Reyes et al., 2010). To test its gene silencing potential, K299 cells were treated with Apt-NS/siRNA only (without drug payload) for 48 hrs, and stained with anti-ALK antibody to detect cellular ALK proteins. Flow cytometry analysis revealed that treatment with Apt-NS/siRNA significantly decreased cellular ALK protein level (FIG. 4A). In the control experiment, conventional lipofectamine-mediated transfection with equimolar amount of siRNA was conducted and resulted in a mild effect on cellular ALK protein expression, compared to untreated cells. In addition, cells were also stained with acridine orange/ethidium bromide (AO/EB) to highlight apoptotic and dead cells and were examined by flow cytometry. As shown in FIG. 4B, treatment with Apt-NS/siRNA resulted in a significantly higher (%) increase in dead and apoptotic cells than lipofectamine-mediated transfection but had no effect on control U937 cells.

To evaluate therapeutic potential, cultured cells were treated with Apt-NS/DOX, carrying DOX payload at final concentration 0.25 for 24 hrs. Cells were then collected and stained with AO/EB to highlight viable (green) and dead (red) cells. Fluorescence microscopy studies revealed that treatment with Apt-NS/DOX induced a significantly higher death rate of K299 cells than the control groups treated with equimolar amount of free DOX or without treatment (FIG. 4C). Importantly, the Apt-NS/DOX had minimal effect on control U937 cells, although they showed similar sensitivity to free DOX treatment, which suggests fewer off-target effects.

To validate potential combination therapeutic effects and rule out non-specific toxicity, cultured cells were exposed to different treatments, as illustrated in FIG. 4D. In particular, the cells were treated with the nanostructure core (NS), nanostructure carrying DOX only (NS/DOX), nanostructure carrying siRNA only (NS/siRNA), nanostructure carrying both DOX and siRNA (NS/DOX-siRNA), Apt-NS, Apt-NS/DOX, Apt-NS/siRNA, Apt-NMed, and an equimolar amount of free DOX (0.25 μM final concentration). Cells were collected 48-hrs' post-treatment and stained with AO/EB to highlight apoptotic and dead cells. Flow cytometry analysis revealed that treatment with free DOX exhibited similar toxicity to both K299 and U937 cells, while treatment with NS/DOX showed no toxic effect to the cells due to lack of free DOX in the cultures (FIG. 4D). However, treatment with Apt-NS/DOX specifically induced death of K299 cells (from 6.5% at baseline to 21% death rate), but had no toxicity against control U937 cells. More importantly, treatment with Apt-NMed further increased the death rate of K299 cells (up to 28%), thus showing the combined effects of targeted chemotherapy, via released DOX, and gene therapy through ALK-specific siRNA on ALK ALCL cells only.

### Apt-NMed Treatment Inhibited ALCL Tumor Growth and Improved Mouse Survival

For therapeutic study of ALCL, mouse model bearing ALCL and CD30 (−) control tumors, derived from K299 cells and MDA-MB-231 breast cancer cells, respectively, was established. Development of both xenograft tumors in the same mouse was monitored by bioluminescence imaging (FIG. 5A), given that tumor cells stably expressed luciferase. To validate targeted delivery, Apt-NS/siRNA was conjugated to the near-infrared fluorescent reporter IRD800CW (for tracking purposes), and systemically administrated through the tail veins of tumor-bearing mice (6.7 μg/mouse). Whole body optical imaging was performed using the IVIS 200 Imaging System immediately post administration. In comparison to tumor signals recorded by bioluminescence, Apt-NS/siRNA selectively highlighted ALCL tumor, but did not react with control tumor in the same mouse (FIG. 5A). Quantitative imaging analysis revealed that imaging signal at ALCL tumor site was 5-fold higher than that detected in control tumor and 8-fold higher than body background. For confirmation, tumor tissues were removed from the mice, and re-scanned ex vivo (FIG. 5B). As expected, in contrast to breast cancer tumor, ALCL tumor fluoresced due to Apt-NS/siRNA, but both tumors emitted bioluminescent radiation. In addition, fresh tumor cells were collected and examined under fluorescence microscope. The ALCL tumor cells (K299) were specifically highlighted by Apt-NS/siRNA, but no signal was detected in breast cancer tumor cells MDA-MB-231. Histological and immunohistochemical studies of tumor tissues demonstrated expression of CD30 in ALCL tumors (FIG. 5C). To evaluate biostability, kinetic imaging was conducted at different time points, post-systemic administration of Apt-NS/siRNA. As demonstrated in FIG. 5D, selective accumulation of Apt-NS/siRNA in ALCL tumor was detected immediately post administration; the signal lasted up to 8 hrs, indicating the capacity of Apt-NS/siRNA for drug delivery and therapeutic use.

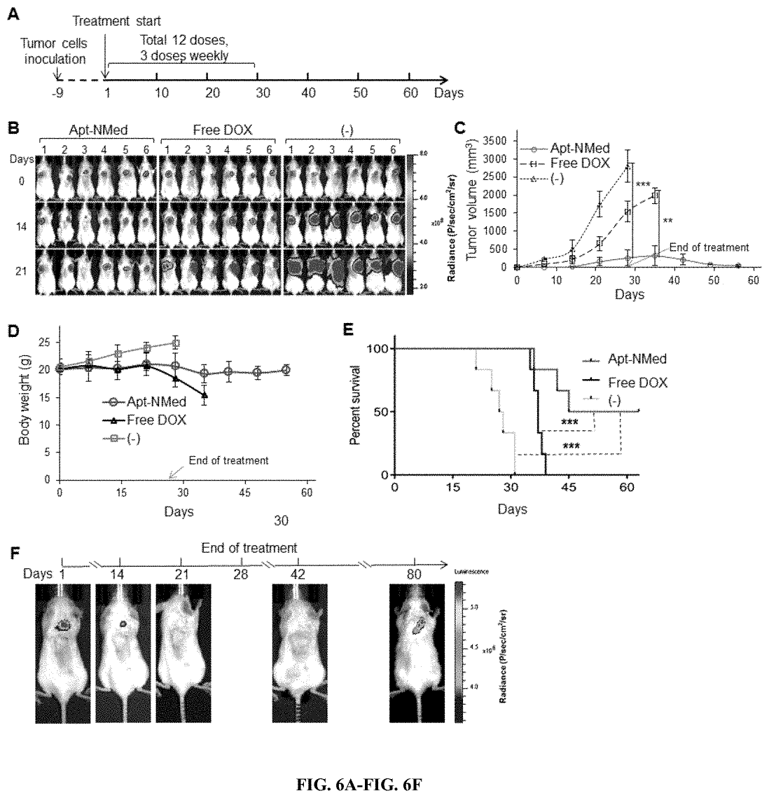

To study the therapeutic potential, xenograft mice were generated, and randomly assigned to receive different treatments (6 mice/group). As illustrated in FIG. 6A, mice were systemically administered Apt-NMed (0.24 mg/mouse carrying 20 μg of DOX payload), equimolar amount of free DOX (20 μg/mouse), or vehicle alone as an untreated control (−), three times per week for a total 12 doses. Tumor growth was monitored by whole body bioluminescence, and tumor cell signals were recorded every 7 days (FIG. 6B). Changes in tumor volume were then calculated and graphed. As depicted in FIG. 6C, at the endpoint of treatment (day 28), the average tumor volume in the Apt-NMed-treated group was 10-fold lower than the untreated control group (−) and 6-fold lower than free DOX-treated group, findings which corroborate the high therapeutic efficacy of Apt-NMed.

To evaluate non-specific toxic effects of treatment, the body weights of the mice were measured every 7 days and graphed. As shown in FIG. 6D, treatment with Apt-NMed resulted in insignificant changes in mice body weight, suggesting minimal toxicity in mice. In contrast, treatment with free DOX induced a considerable decrease in mouse body weight with 20% loss at day 35, consistent with severe side effects of DOX. Notably, the body weight of untreated mice increased due to rapid growth of tumor masses (FIG. 6D). To study survival rate, mice were observed for 65 days post first dose treatment (FIG. 6E). All the mice died within 31 days in the untreated group. In contrast, treatment with free DOX slightly prolonged survival of tumor-bearing mice, with the last mouse dying at day 38. Interestingly, treatment with Apt-NMed significantly improved mouse survival, as shown by the 50% survival rate at 65 days. More importantly, the tumor masses in surviving mice had regressed after completion of treatment, and these mice were tumor-free at the endpoint. After treatment was stopped, the tumors slowly recurred (FIG. 6C and FIG. 6F).

### CONCLUSION

A new method to form nanomedicine through simple self-assembly and self drug-loading has been described herein. This nanomedicine was formulated by self-assembly of synthetic oligonucleotides containing IgG heavy chain derived single stranded DNA sequences, cancer cell-targeting aptamer sequences and oncogene-specific siRNA, followed by self-loading of the chemotherapeutic drug. It allows for targeted delivery, which leads to specific accumulation in the tumor, which is treated simultaneously with chemotherapy and oncogene-specific gene therapy. This therapy has higher therapeutic efficacy than single drug therapy and lower side effects than equal amounts of drug therapy. This nanomedicine can be adapted to treat different cancers by simple modification of the design. IgG sequences were selected to ensure biosafety.

A specific treatment for anaplastic large cell lymphoma that consists of single stranded DNA. derived from the IgG heavy chain, ALK siRNA, CD30 aptamer and doxorubicin.

A multifunctional aptamer-nanomedicine that can simultaneously target chemotherapy and gene therapy was developed and is depicted by the schematic shown in FIG. 1A. This nanomedicine is formulated by self-assembly of synthetic oligonucleotides consisting of complementary sequences adapted from human IgG heavy chain, a CD30-specific aptamer and anaplastic lymphoma kinase (ALK)-specific siRNA followed by self-loading of the chemotherapeutic drug doxorubicin.

An exemplary Aptamer-Nanomedicine composition included the following:

(a) ssDNA: synthetic complementary sequences that were adapted from human IgG heavy chain (NCBI database: GenBank: X82593.1);

(b) CD30 aptamer. (Anaplastic large cell lymphoma and Hodgkin's lymphoma cells that express high levels of CD30);

(c) ALK siRNA (ALK encodes the protein ALK receptor kinase, which is involved in signaling pathways that are important for cell growth, proliferation and differentiation (Alterations in this gene are associated with neuroblastomas, lung cancer, anaplastic large cell lymphoma, and inflammatory myofibroblastic tumor); and

(d) Doxorubicin (an injected chemotherapy agent that is used to treat cancers, including breast cancer, bladder cancer, Kaposi's sarcoma, lymphoma, and acute lymphocytic leukemia).

The single-stranded DNA sequences for aptamer nanostructure formation are as follows:

ssDNA #1—CD30 aptamer/ssDNA/CD30 aptamer/ssDNA
(SEQ ID NO: 1)
5′-ACTGGGCGAAACAAGTCTATTGACTATGAGCGGTAAAGCTTGTGTAG
AGAGaaaACTGGGCGAAACAAGTCTATTGACTATGAGCaaaTGGATCCAG
GACTAGTAAAGC-3′.
ssDNA #2—ssDNA/ssDNA/ssDNA
(SEQ ID NO: 2)
5′-CTCTCTACACAAGCTTTACCaaAAAAAAAAAAAAAAAAATTaaGCTT
TACTAGTCCTGGATCCA-3′.
ssDNA/siRNA #3—CD30 aptamer/ssDNA/siRNA
(SEQ ID NO: 3)
5′-ACTGGGCGAAACAAGTCTATTGACTATGAGaaAATTTTTTTTTTTTT
TTTTaaCACUUAGUAGUGUACCGCC-3′.
Antisense anti-ALK siRNA
(SEQ ID NO: 4)
5′-GGCGGUACACUACUAAGUG-3′

### REFERENCES

The following references, to the extent that they provide exemplary procedural or other details supplementary to those set forth herein, are specifically incorporated herein in their entirety by express reference thereto:
AGUDELO, D et al., “Intercalation of antitumor drug doxorubicin and its analogue by DNA duplex: structural features and biological implications,” Int. J. Biol. Macromol., 66:144-150 (February 2014).
ALTSCHUL, S F et al., “Gapped BLAST and PSI-BLAST: a new generation of protein database search programs,” Nucl. Acids Res., 25(17):3389-3402 (September 1997).
ANGELL, C et al., “DNA Nanotechnology for precise control over drug delivery and gene therapy,” Small, 12(9):1117-1132 (January 2016).
AUFFAN, M et al., “Towards a definition of inorganic nanoparticles from an environmental, health and safety perspective,” Nat. Nanotechnol., 4(10):634-641 (September 2009).
BANERJEE, J and NILSEN-HAMILTON, M, “Aptamers: multifunctional molecules for biomedical research,” J. Mol. Med. (Berl)., 91(12):1333-1342 (September 2013).
CHEN, K et al., “Advances in the development of aptamer drug conjugates for targeted drug delivery,” Wiley Interdiscip. Rev. Nanomed. Nanobiotechnol., 9(3):e1438 (October 2016).
CHEN, Y J et al., “DNA nanotechnology from the test tube to the cell,” Nat. Nanotechnol., 10(9):748-760 (September 2015).
DE GOEIJ, B E and LAMBERT, J M, “New developments for antibody-drug conjugate-based therapeutic approaches,” Curr. Opin. Immunol., 40:14-23 (March 2016).
DE PUIG, H et al., “Selective light-triggered release of DNA from gold nanorods switches blood clotting on and off,” PLoS One, 8(7):e68511 (July 2013).
DENG, B et al., “Aptamer binding assays for proteins: the thrombin example—a review,” Anal. Chim. Acta., 837:1-15 (May 2014).
DIEFENBACH, C S and LEONARD, J P, American Society of Clinical Oncology Educational Book, American Society of Clinical Oncology Meeting, 2012:162 (2012).
FAROKHZAD, O C et al., “Targeted nanoparticle-aptamer bioconjugates for cancer chemotherapy in vivo,” Proc. Nat'l. Acad. Sci. USA, 103(16):6315-6320 (April 2006).
FISHER, R I et al., “Comparison of a standard regimen (CHOP) with three intensive chemotherapy regimens for advanced non-Hodgkin's lymphoma,” N. Engl. J. Med., 328(14):1002-1006 (April 1993).
FRIEDMAN, A D et al., “The smart targeting of nanoparticles,” Curr. Pharm. Des., 19(35):6315-6329 (2013).
GALKIN, A V et al., “Identification of NVP-TAE684, a potent, selective, and efficacious inhibitor of NPM-ALK,” Proc. Nat'l. Acad. Sci. USA., 104(1):270-275 (December 2006).
GOPINATH, S C et al., “Cell-targeting aptamers act as intracellular delivery vehicles,” Appl. Microbiol. Biotechnol., 100(16):6955-6969 (June 2016).
GUO, S et al., “Specific delivery of therapeutic RNAs to cancer cells via the dimerization mechanism of phi29 motor pRNA,” Hum. Gene Ther., 16(9):1097-1109 (September 2005).
HALE, W G, and MARGHAM, J P, “HARPER COLLINS DICTIONARY OF BIOLOGY,” HarperPerennial, New York (1991).
HAQUE, F et al., “Ultrastable synergistic tetravalent RNA nanoparticles for targeting to cancers,” Nano Today, 7(4):245-257 (August 2012).
HARDMAN, J G, and LIMBIRD, L E, (Eds.), “GOODMAN AND GILMAN'S THE PHARMACOLOGICAL BASIS OF THERAPEUTICS” 10^{th} Edition, McGraw-Hill, New York (2001).
HE, X et al., “ATP-responsive controlled release system using aptamer-functionalized mesoporous silica nanoparticles,” Langmuir, 28(35):12909-12915 (August 2012).
HOU, K K et al., “Mechanisms of nanoparticle-mediated siRNA transfection by melittin-derived peptides,” ACS Nano., 7(10):8605-8615 (September 2013).
HSU, F Y et al., “Downregulation of NPM-ALK by siRNA causes anaplastic large cell lymphoma cell growth inhibition and augments the anti cancer effects of chemotherapy in vitro,” Cancer Invest., 25(4):240-248 (June 2007).
HUANG, F et al., “Self-assembled hybrid nanoparticles for targeted co-delivery of two drugs into cancer cells,” Chem. Commun. (Camb)., 50(23):3103-3105 (March 2014).
JIANG, D et al., “DNA nanomaterials for preclinical imaging and drug delivery,” J. Control. Release, 239:27-38 (August 2016).
JO, H and BAN, C, “Aptamer-nanoparticle complexes as powerful diagnostic and therapeutic tools,” Exp. Mol. Med., 48:e230 (May 2016).
KYTE, J, and DOOLITTLE, R F, “A simple method for displaying the hydropathic character of a protein,” J. Mol. Biol., 157(1):105-132 (May 1982).
LEE, H et al., “Molecularly self-assembled nucleic acid nanoparticles for targeted in vivo siRNA delivery,” Nat. Nanotechnol., 7(6):389-393 (June 2012).
LEVY-NISSENBAUM, E et al., “Nanotechnology and aptamers: applications in drug delivery,” Trends Biotechnol., 26(8):442-449 (June 2008).
LI, L et al., “Nucleolin-targeting liposomes guided by aptamer AS1411 for the delivery of siRNA for the treatment of malignant melanomas,” Biomaterials, 35(12):3840-3850 (January 2014).
LIU, J et al., “Multifunctional aptamer-based nanoparticles for targeted drug delivery to circumvent cancer resistance,” Biomaterials, 91:44-56 (March 2016).
MA, H and ABDUL-HAY, M “T-cell lymphomas, a challenging disease: types, treatments, and future,” Int. J. Clin. Oncol., 22(1):18-51 (October 2016).
MCKEAGUE, M and DEROSA, MC, “Challenges and opportunities for small molecule aptamer development,” J. Nucleic Acids, 2012:748913 (October 2012).
MEYER, C et al., “Cell-specific aptamers as emerging therapeutics,” J. Nucleic Acids, 2011:904750 (August 2011).
NEEDLEMAN, S B and WUNSCH, C D, “A general method applicable to the search for similarities in the amino acid sequence of two proteins,” J. Mol. Biol., 48(3):443-453 (March 1970).
PALA, K et al., “Tumor-specific hyperthermia with aptamer-tagged superparamagnetic nanoparticles,” Int. J. Nanomedicine, 9:67-76 (December 2013).
PAREKH, P et al., “Immunotherapy of CD30-expressing lymphoma using a highly stable ssDNA aptamer,” Biomaterials, 34(35):8909-8917 (August 2013).
PEREZ-ARNAIZ, C et al., “New insights into the mechanism of the DNA/doxorubicin interaction,” J. Phys. Chem. B., 118(5):1288-1295 (January 2014).
PIERCE, J M and MEHTA, A, “Diagnostic, prognostic and therapeutic role of CD30 in lymphoma,” Expert Rev. Hematol., 10(1):29-37 (December 2016).
PORCIANI, D et al., “Aptamer-mediated codelivery of Doxorubicin and NF-κB decoy enhances chemosensitivity of pancreatic tumor cells,” Mol. Ther. Nucleic Acids, 4:e235 (April 2015).
REYES-REYES, E M et al., “A new paradigm for aptamer therapeutic AS1411 action: uptake by macropinocytosis and its stimulation by a nucleolin-dependent mechanism,” Cancer Res., 70(21):8617-8629 (September 2010).
SCHIRRMANN, T et al., “CD30 as a therapeutic target for lumphoma,” BioDrugs, 28(2): 181-209 (April 2014).
SHIAO, Y S et al., “Aptamer-functionalized gold nanoparticles as photoresponsive nanoplatform for co-drug delivery,” ACS Appl. Mater. Interfaces, 6(24):21832-21841 (June 2014).
SHU, D et al., “Thermodynamically stable RNA three-way junction for constructing multifunctional nanoparticles for delivery of therapeutics,” Nat. Nanotechnol., 6(10):658-667 (September 2011).
SINGLETON, P and SAINSBURY, D, “DICTIONARY OF MICROBIOLOGY AND MOLECULAR BIOLOGY,” 2^{nd} Ed., John Wiley and Sons, New York (1987).
SUN, H and ZU, Y, “Aptamers and their applications in nanomedicine,” Small, 11(20):2352-2364 (February 2015).
SUN, H et al., “Aptamers: versatile molecular recognition probes for cancer detection,” Analyst, 141(2):403-415 (January 2016).
TAGHDISI, S M et al., “Reversible targeting and controlled release delivery of daunorubicin to cancer cells by aptamer-wraped carbon nanotubes,” Eur. J. Pharm. Biopharm., 77(2):200-206 (December 2010).
TIGLI, R. S. Aydin, “Drug targeting systems for cancer therapy: nanotechnological approach,” Mini. Rev. Med. Chem., 14(13):1048-1054 (2015).
TOY, R et al., “Targeted nanotechnology for cancer imaging,” Adv. Drug Deliv. Rev., 76:79-97 (August 2014).
TURTURRO, F et al., “Model of inhibition of the NPM-ALK Kinase activity by Herbimycin A,” Clin. Cancer Res., 8(1):240-245 (January 2002).
WANDTKE, T et al., “Aptamers in diagnostics and treatment of viral infections,” Viruses, 7(2):751-780 (February 2015).
WANG, J et al., “Aptamer-conjugated nanorods for targeted photothermal therapy of prostate cancer stem cells,” Chem. Asian 8(10):2417-2422 (June 2013).
WANG, J et al., “Photosensitizer-gold nanorod composite for targeted multimodal therapy,” Small, 9(21):3678-3684 (May 2013).
WU, J et al., “Nucleolin targeting AS1411 modified protein nanoparticle for antitumor drugs delivery,” Mol. Pharm., 10(10):3555-3563 (August 2013).
XING, H et al., “Selective delivery of an anticancer drug with aptamer-functionalized liposomes to breast cancer cells in vitro and in vivo,” J. Mater. Chem. B., 1(39):5288-5297 (October 2013).
ZHANG, L et al., “Co-delivery of hydrophobic and hydrophilic drugs from nanoparticle-aptamer bioconjugates,” ChemMedChem., 2(9): 1268-1271 (September 2007).
ZHAO, N et al., “A nanocomplex that is both tumor cell-selective and cancer gene-specific for anaplastic large cell lymphoma,” J. Nanobiotechnol., 9:2 (January 2011).
ZHAO, N et al., “An ultra pH-sensitive and aptamer-equipped nanoscale drug-delivery system for selective killing of tumor cells,” Small, 9(20):3477-3484 (April 2013).
ZHOU, J and ROSSI, J J, “Cell-specific aptamer-mediated targeted drug delivery,” Oligonucleotides, 21(1): 1-10 (December 2010).
ZHOU, J et al., “Dual functional RNA nanoparticles containing phi29 motor pRNA and anti-gp120 aptamer for cell-type specific delivery and HIV-1 inhibition,” Methods, 54(2):284-294 (January 2011).
ZHU, C L et al., “Bioresponsive controlled release using mesoporous silica nanoparticles capped with aptamer-based molecular gate,” J. Am. Chem. Soc., 133(5):1278-1281 (January 2011).
ZHU, G et al., “Self-assembled, aptamer-tethered DNA nanotrains for targeted transport of molecular drugs in cancer theranostics,” Proc. Nat'l. Acad. Sci. USA, 110(20):7998-8003 (April 2013).
ZHU, J et al., “Progress in aptamer-mediated drug delivery vehicles for cancer targeting and its implications in addressing chemotherapeutic challenges,” Theranostics, 4(9):931-944 (July 2014).
ZHU, Z et al., “Regulation of singlet oxygen generation using single-walled carbon nanotubes,” J. Am. Chem. Soc., 130(33):10856-10857 (July 2008).
ZIMBRES, F M et al., “Aptamers: novel molecules as diagnostic markers in bacterial and viral infections,” Biomed. Res. Int., 2013:731516 (September 2013).
ZUNINO, F et al., “The interaction of daunorubicin and doxorubicin with DNA and chromatin,” Biochim. Biophys. Acta., 607(2):206-214 (April 1980).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising,” “having,” “including,” or “containing” with reference to an element or elements is intended to provide support for a similar aspect or embodiment of the to invention that “consists of” “consists essentially of” or “substantially comprises” the particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved.

All such similar substitutes and modifications apparent to those skilled in the art are deemed to be within the spirit, scope and concept of the invention as defined by the appended claims.

What is claimed is:

1. A method of providing treatment or amelioration of at least one symptom of a mammalian disease or cancer, comprising administering to a mammalian subject in need thereof, an effective amount of a composition comprising a population of self-assembling, cancer cell-specific aptamer-containing nanoparticles sufficient to provide treatment or amelioration of at least symptom of the disease or the cancer in the mammal.

2. A method of improving the outcome of conventional chemotherapeutic treatment of a mammalian cancer, comprising administering to a mammalian subject in need thereof, an effective amount of a composition that comprises: a population of self-assembling nanoparticles comprising: (a) at least one cancer cell-specific aptamer sequence; (b) a population of anti-ALK siRNAs; and (c) at least one chemotherapeutic agent, wherein the composition is administered to the mammal in an amount and for a time effective to improve the outcome as compared to treatment with the chemotherapeutic agent alone.

3. A method of increasing the life expectancy of a patient undergoing treatment for cancer, comprising administering to the patient, a composition that comprises: a population of self-assembling nanoparticles comprising: (a) at least one cancer cell-specific aptamer sequence; (b) a population of anti-ALK siRNAs; and (c) at least one chemotherapeutic agent, wherein the composition is administered to the patient in an amount and for a time effective to increase the life expectancy of the patient undergoing treatment for the cancer.

4. The method of claim 1, claim 2, or claim 3, wherein the cancer is diagnosed as, or is identified as, a refractory, a metastatic, a relapsed, or a treatment-resistant cancer.

5. The method of claim 1, claim 2, or claim 3, wherein the cancer is anaplastic large cell lymphoma.

6. The method of claim 1, claim 2, or claim 3, wherein the composition is administered orally to the mammal, either as a single dose, or as a series of multiple doses over a period of several days, several weeks, or several months or longer.

7. The method of claim 1, claim 2, or claim 3, wherein the chemotherapeutic agent is selected from the group consisting of cyclophosphamide, doxorubicin, 5-fluorouracil, docetaxel, paclitaxel, trastuzumab, methotrexate, epirubicin, cisplatin, carboplatin, vinorelbine, capecitabine, gemcitabine, mitoxantrone, isabepilone, eribulin, lapatinib, carmustine, a nitrogen mustard, a sulfur mustard, a platin tetranitrate, vinblastine, etoposide, camptothecin, and combinations thereof.

8. The method of claim 6, wherein the chemotherapeutic is doxorubicin.

9. The method of claim 1, claim 2, or claim 3, wherein the cancer cell-specific aptamer sequence is a CD30-specific aptamer sequence.

10. The method of claim 1 or claim 2, wherein the mammal is human.

11. The method of claim 1, claim 2, or claim 3, wherein the composition further comprises at least one agent selected from the group consisting of an immunomodulating agent, a neuroactive agent, an anti-inflammatory agent, an anti-lipidemic agent, a hormone, a receptor agonist, a receptor antagonist, an anti-infective agent, a protein, a peptide an antibody, an antigen-binding fragment, an enzyme, an RNA, a DNA, an siRNA, an mRNA, a ribozyme, a hormone, a cofactor, a steroid, an antisense molecule, and any combination thereof.

12. The method of claim 9, wherein the CD30-specific comprise an isolated nucleic sequence that is at least 98% homologous to the sequence of any one of SEQ ID NO:1, SEQ ID NO:2, SEQ ID NO:3, and SEQ ID NO:4.

13. The method of claim 2 or claim 3, wherein the ALK-specific siRNAs comprise an isolated nucleic sequence that is at least 98% homologous to the sequence of SEQ ID NO:4.

14. A composition for treating cancer in a mammal subject, comprising:
(a) a plurality of synthetic ssDNA sequences that are complementary to at least a first sequence region that encodes a human IgG heavy chain;
(b) a population of CD30 aptamer-specific nucleic acids;
(c) a population of ALK receptor kinase-specific siRNAs; and
(d) at least one chemotherapeutic agent.

15. The composition of claim 14, wherein the chemotherapeutic agent comprises doxorubicin.

16. The composition of claim 14, further comprising: (e) a pharmaceutically-acceptable buffer, diluent, carrier, or vehicle, suitable for parenteral administration to a human.

17. The composition of claim 14, formulated for administration to a human diagnosed with anaplastic large cell lymphoma (ALCL).


SEQUENCE LISTING (4 sequences)

SEQ ID NO: 1 - DNA, 109 bp, Homo sapiens
actgggcgaaacaagtctattgactatgagcggtaaagcttgtgtagagagaaaactgggcgaaacaagtctattgactatgagcaaatggatccaggactagtaaagc

SEQ ID NO: 2 - DNA, 64 bp, Homo sapiens
ctctctacacaagctttaccaaaaaaaaaaaaaaaaaaattaagctttactagtcctggatcca

SEQ ID NO: 3 - DNA, 72 bp, Homo sapiens
actgggcgaaacaagtctattgactatgagaaaatttttttttttttttttaacacuuaguaguguaccgcc

SEQ ID NO: 4 - RNA, 19 nt, Homo sapiens
ggcgguacacuacuaagug